(19) United States
(12) Patent Application Publication
(10) Pub. No.: US 2003/0200032 A1
(43) Pub. Date: Oct. 23, 2003

(54) Determination of compatibility of a set chemical modifications with an amino-acid chain

(75) Inventors: Sean P. Keating, San Mateo, CA (US); Heinz Breu, Palo Alto, CA (US)

Correspondence Address: MILA KASAN, PATENT DEPT., APPLIED BIOSYSTEMS, 850 LINCOLN CENTRE DRIVE, FOSTER CITY, CA, 94404, US

(73) Assignee: Applera Corporation, Foster City, CA

(21) Appl. No.: 10/378,694

(22) Filed: Mar. 3, 2003

Related U.S. Application Data
(60) Provisional application No. 60/361,222, filed on Mar. 1, 2002.
(60) Provisional application No. 60/361,791, filed on Mar. 4, 2002.

(51) Int. Cl. G01N 33/48

(52) U.S. Cl. 702/19

(57) ABSTRACT

Peptide mass mapping is a technique whereby masses determined from mass spectrometry of a protein digest are compared to the masses of theoretical peptides derived from a reference protein, specified as an amino-acid sequence. In some cases differences between experimental and theoretical masses can be accounted for by chemical modifications of the actual protein with respect to the reference, often as a result of post-translational modification (PTM). Typically such modifications are applicable to specific sets of amino-acid residues. Analysis of these mass differences can therefore lead to identification of PTMs. In various cases, it is desirable that such analysis in general allow for the possibility of a peptide having several different PTMs, and furthermore it is desirable in various cases that the chemical compatibility of a putative combination of PTMs with the peptide sequence be verified. Embodiments are described herein wherein compatibility verification is formulated as a problem in graph theory. Theory and implementation of a solution are discussed and described.

### RELATED APPLICATIONS

This application claims priority from U.S. Provisional Application No. 60/361,791, filed on Mar. 4, 2002 and from U.S. Provisional Application No. 60/361,222, filed Mar. 1, 2002; each of which is incorporated herein by reference.

### FIELD

The present teachings relate to systems and methods to determine and verify the compatibility of chemical modifications with an amino acid chain.

### REFERENCES

Boost Graph Library (http://www.boost.org/libs/graph/doc/index.html).

Papadimitriou & Steiglitz (1984), Combinatorial Optimization: Algorithms and Complexity.

Sedgewick, R. (1988), Algorithms.

Skiena, S. S. (1998), The Algorithm Design Manual.

### INTRODUCTION

Peptide mass mapping is a technique whereby masses determined from mass spectrometry of a protein digest are compared to the masses of theoretical peptides derived from a reference protein, specified as an amino-acid sequence. In some situations, differences between experimental and theoretical masses can be accounted for by chemical modifications of the actual protein with respect to the theoretical. These modifications are often a result of one or more post-translational modifications (PTMs). Typically such modifications are applicable to specific amino-acid residues or sets of amino-acid residues. Analysis of these mass differences can therefore lead to identification of potential PTMs that may be compatible with a particular peptide. Accordingly, it is desirable that such analysis in general allow for the possibility of a peptide having several different PTMs, and furthermore it is desirable to verify that a putative PTM set and the peptide sequence are chemically compatible.

### SUMMARY

Various embodiments of the present teachings provide systems and methods for determining and verifying the compatibility of chemical modifications with a biopolymer, such as an amino acid chain.

According to various embodiments, compatibility verification is formulated as a problem in graph theory. Theory and implementation of a solution are discussed and described herein.

Various embodiments of the present teachings provide a system that applies graph theory, e.g., maximum cardinality matching in a bipartite graph, to determine chemical compatibility of an amino-acid chain with a set of chemical modifications.

Other embodiments include methods for use in peptide mass mapping to identify post-translational modifications, including measuring the molecular weight of a peptide fragment, comparing that measured molecular weight to a molecular weight expected for an unmodified fragment having the same sequence, thereby ascertaining a difference from an unmodified fragment, and applying a graph theory formulation to determine compatibility between the measured molecular weight and a set of possible post-translational modifications.

Still other embodiments include methods wherein the graph theory formulation includes maximum cardinality matching in a bipartite graph.

Other embodiments include methods to determine compatibility between an amino-acid residue chain having an experimentally-ascertained molecular weight and a known amino acid sequence and a set of post-translational chemical modifications, including constructing a bipartite graph comprising a vertex for each residue, a vertex for each modification, and an edge for each compatible pair; and seeking a maximum cardinality matching comprising a set of edges (i) wherein no two edges share a vertex, and (ii) wherein every modification is paired with a residue.

Further embodiments include methods to determine chemical compatibility of an amino-acid residue chain with a set of chemical modifications, which include constructing a graph, finding a maximum cardinality matching, and determining whether the cardinality is equal to the number of modifications.

Various aspects also relate to a method for applying a graph theory formulation to determine chemical compatibility of an amino-acid residue chain with a set of chemical modifications. In certain embodiments, the graph theory formulation includes maximum cardinality matching in a bipartite graph.

Further aspects relate to a process of determining chemical compatibility of an amino-acid residue chain with a set of possible chemical modifications. In various embodiments, the process includes constructing a bipartite graph having a vertex for each residue, a vertex for each modification, and an edge for each compatible pair. The process then seeks a maximum cardinality match of a set of edges (i) wherein no two edges share a vertex, and (ii) wherein every modification is paired with a residue.

Additional aspects relate to a method to determine chemical compatibility of an amino-acid residue chain with a set of chemical modifications, including: constructing a graph, finding a maximum cardinality match, and determining whether the cardinality (number of edges) is equal to the number of modifications. In certain embodiments, the maximum cardinality match is found by selecting any match (an empty one is valid and convenient), finding an augmenting path and then using this path to define a new match. This process is then repeated until no additional path can be found.

Various aspects related to methods for peptide analysis, include comparing a measured mass of an analyte peptide against the masses of theoretical peptides derived from a reference protein, and applying a graph theory formulation to determine the chemical compatibility of a selected set of post-translational modifications (PTMs) with the theoretical peptides, whereby a set of candidate peptides is developed, having one or more peptides, including one or more peptides bearing one or more PTMs, having a mass like or similar to that of said analyte peptide.

According to various embodiments, the measured mass of the analyte peptide is determined by mass spectrometry of a protein digest. Further aspects relate to program storage devices readable by a machine, embodying a program of instructions executable by the machine to perform method steps for peptide analysis. In various embodiments, the method steps include (i) comparing a measured mass of an analyte peptide against the masses of theoretical peptides derived from a reference protein, and (ii) applying a graph theory formulation to determine the chemical compatibility of a selected set of post-translational modifications (PTMs) with the theoretical peptides, whereby a set of candidate peptides is developed, having one or more peptides, including one or more peptides bearing one or more PTMs, having a mass like or similar to that of said analyte peptide.

In various embodiments, the graph theory formulation includes maximum cardinality matching in a bipartite graph.

Additional aspects relate to program storage devices readable by a machine, embodying a program of instructions executable by the machine to perform method steps for use in peptide analysis. In various embodiments, the method steps include applying a graph theory formulation to determine chemical compatibility of an amino-acid residue chain with a set of chemical modifications.

In certain embodiments, the graph theory formulation includes maximum cardinality matching in a bipartite graph.

According to various embodiments, the chemical modifications include post-translational modifications.

In accordance with various embodiments, the method steps further include providing output relating a measured peptide mass with a theoretic peptide having some chemical modification or set of chemical modifications.

Various embodiments include a computer systems that selects a set of candidate sets of post-translational modifications based on the difference between measured parameters of tan analyte peptide and a theoretical peptide.

Further aspects relate to methods in a computer system for analysis of an analyte peptide, including receiving an input having a measured mass of an analyte peptide, presenting to a user a listing including a plurality of post-translational modifications (PTMs), receiving from said user a user-selected set selected from said plurality of PTMs, and presenting to said user one or more theoretical peptides, bearing one or more PTMs from said user-selected set that have been checked for chemical compatibility with said theoretical peptides, having a mass like or similar to that of said analyte peptide within a defined mass tolerance.

According to certain embodiments, mapping matches the molecular weight of the peaks found in a data file (e.g., from a mass spectrograph) to the molecular weight of peptides predicted from a sequence of a known protein (which, according to the present teachings, may include compatibility-checked chemical modifications). If the molecular weights match within the mass tolerance, then the identity of the protein used in the study can be confirmed. In various embodiments, the mass tolerance is selectable by a user. For example, a tolerance of +/−5, 10, 25, 50, 100, 500 mass units, or other number, can be selected by a user.

### BRIEF DESCRIPTION OF THE DRAWINGS

FIG. 1 is a block diagram illustrating an overview of an analysis system used to compare the molecular weight of an actual digested peptide fragment with a corresponding theoretical peptide fragment, select a potential PTM set to account for any weight difference, and verify the selected PTM set's compatibility with the theoretical peptide fragment, according to various embodiments of the present teachings.

FIG. 2 is a flowchart illustrating an overview of a method for comparing the molecular weight of an actual digested peptide fragment with a corresponding theoretical peptide fragment, selecting a potential PTM set to account for any weight difference, and verifying the PTM set's compatibility with the theoretical peptide fragment, according to various embodiments of the present teachings.

FIG. 3 illustrates an example of a protein digest using the protease trypsin.

FIG. 4 illustrates a broad overview of one method of peptide mapping.

FIG. 5 illustrates the results of O-phosphorylation of the amino acid residue Serine (S).

FIG. 6 illustrates a bipartite graph used to verify whether the PTM set {Ph, Su} is compatible with the amino acid sequence: Tyrosine, Isoleucine, Proline, Glycine, Threonine, Lysine (YIPGTK).

FIG. 7 illustrates a method of finding a maximum cardinality match using an augmenting path, according to various embodiments of the present teachings.

FIG. 8 illustrates a user interface for choosing a user-defined set of post-translational modifications.

### DEFINITIONS

Analyte peptide—An analyte peptide is a peptide undergoing identification and characterization. Identification can include but is not limited to the determination of its mass, sequence, its protein of origin, and any modification that it may have undergone.

Bipartite graph—A graph with only two kinds vertices and the edges are only allowed between nodes of the different kinds.

Chemical compatibility—When used in the context of a peptide and a set of post-translational modifications, this term signifies that each post-translational modification in a set of post-translational modifications can be assigned to different amino acid in a peptide fragment so that the chemical compatibility rules specifying the modifications that an amino acid can undergo are satisfied. When used in the context of a single post-translational modification and a single amino acid, this term signifies that the amino acid in question can undergo the modification in question.

Correspondence—When used in the context of two peptide fragments signifies that a reference peptide fragment has the same amino acid sequence as a peptide fragment of interest.

Compatibility (See Chemical Compatibility)

Peptide (mass) fingerprinting (PMF)—The most commonly used strategy for protein identification by mass spectrometry is Peptide Mass Fingerprinting. The target protein is digested with a proteolytic enzyme such as trypsin and the mass spectrometer measures accurate masses of a few peptides derived from the digest. These masses are compared with a theoretical list of peptide fragments calculated from databases of known protein sequences. The masses of about 4-5 peptides are generally sufficient to identify a protein of known amino acid sequence unambiguously. However as databases of known protein sequences have become larger, the amount of data required to identify a specific protein has increased. Therefore reliable identifications by peptide mass fingerprinting require both an increasing number of peptide masses and highly accurate mass measurements. As well, PMF requires highly accurate identification of the peptides and any post-translational modifications associated with them.

Peptide (mass) mapping—A method to identify an analyte peptide using an algorithm to match said analyte peptide to a theoretical peptide. Matches are generally made on the basis of molecular weight but other characteristics of biomolecules can be used. Often the match is close but not exact and other methods are used to identify the sources of the difference. Post-translational modifications are often the cause of molecular weight mismatches.

Post-translational modification (PTM)—PTMs include any modification that affects a polypeptide or protein during or after translation.

Reference peptide—same as theoretical peptide.

Theoretical peptide—A theoretical peptide is a peptide that is used for comparison to an analyte peptide. It is often compared to an analyte peptide on the basis of molecular weight and sequence composition. Reference peptides can originate from a reference protein or be entities unto themselves without association to a protein. A theoretical peptide for a given protein may be generated by an in silico digestion of the protein.

### DESCRIPTION OF VARIOUS EMBODIMENTS

Proteins account for more than 50% of the dry weight of most cells, and they are instrumental in almost everything cells do. For example, proteins are used for structural support, storage, transportation, signaling, movement, and defense. In addition, as enzymes, proteins selectively accelerate necessary chemical reactions in the cell.

Consistent with their diverse functions, proteins are the most structurally sophisticated macromolecules in a cell. Proteins vary extensively in structure, each type of protein having a unique three-dimensional shape corresponding to their particular function. But as diverse as proteins are individually, they are all polymers constructed from the same set of amino acids, the universal monomers of proteins.

Protein synthesis, or translation, involves the linkage of amino acids by dehydration synthesis to form peptide bonds. The chain of amino acids is also known as a polypeptide. During and after translation, a polypeptide chain begins to coil and fold spontaneously to form a functional protein of specific three dimensional conformation. Some proteins contain only one polypeptide chain while others, such as hemoglobin, contain several polypeptide chains combined together. The sequence of amino acids in each polypeptide or protein is unique to that protein, so each protein has its own, unique three-dimensional shape.

For most proteins, additional steps are required before the protein can begin doing its particular job. Accordingly, certain amino acids of a polypeptide or a protein may be chemically modified during or after translation. As used herein the term “post-translational modification” (PTM) includes any modification that effects a polypeptide or protein during or after translation. There are many types of PTMs. PTMs include, for example, proteolytic cleavage, glycosylation, acylation, methylation, phosphorylation, sulfation, prenylation, hydroxylation, carboxylation, and the like.

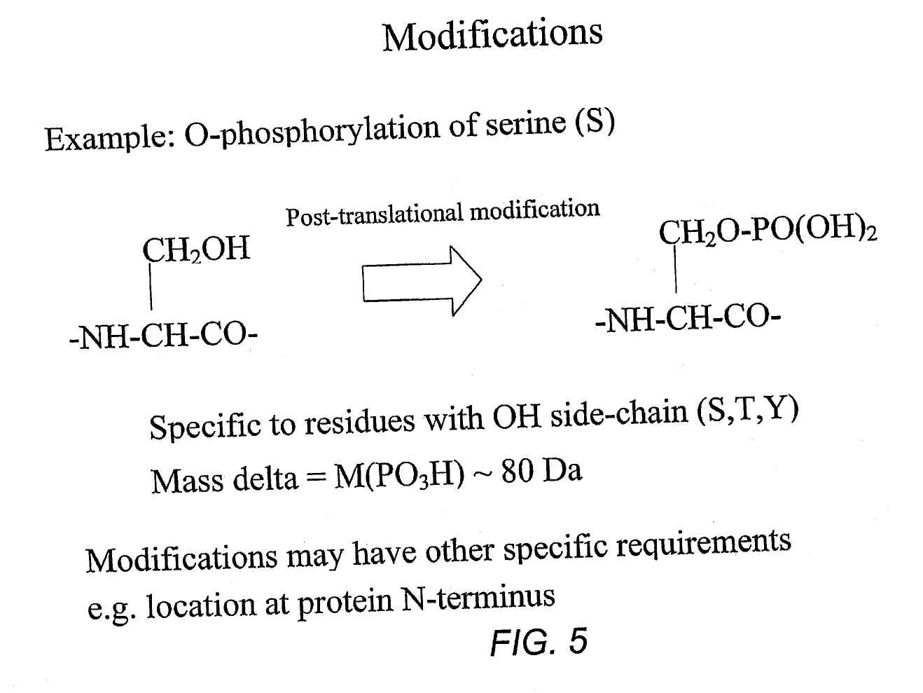

Certain general rules can be applied to PTMs. First of all, any given modification is particular, in that it can only affect specifically defined amino acid residues or amino acid sequences. For example, the modification O-phosphorylation can only apply to amino acid residues with OH side chains: Serine, Threonine, and Tyrosine (S,T,Y). Furthermore, once an amino acid residue has been modified, it will likely not accept another modification. In addition, each particular modification will result in an effective change in the molecular weight of the amino acid sequence. The molecular weight of any given PTM can be readily calculated if it is not known in the art. FIG. 5 illustrates the results of O-phosphorylation of the amino acid residue Serine (S). This particular modification increases the molecular weight of the amino acid by about 80 Daltons.

As those skilled in the art more fully understand the mechanisms underlining post-translational modifications (PTMs) of peptides, the general field of proteomics will be greatly advanced. In particular, skilled artisans will have a greater insight into protein synthesis and its relation to function.

In general, various embodiments of systems and methods described herein are directed to determining whether a given sequence of amino-acid residues can accept a particular set of PTMs. In some limited cases, this problem is not difficult. For example, if only one kind of modification is being considered, then one solution is to simply ascertain whether there are sufficient amino acid residues compatible with that kind of modification. For example, if one was interested only with O-phosphorylation (Ph), which can apply to the amino acid residues S, T, and Y, and the selected PTM set is {Ph, Ph}, then one skilled in the art can readily verify by inspection that the sequence YIPGTK can accept this. (This particular sequence has two available amino acid residues, T and Y, to accept each of the Ph modifications).

If any modification in the PTM set cannot be applied to any residue in the sequence, or there are more modifications than available residues, then the PTM set is not compatible. As an example, that illustrates the general problem, consider the PTM set {Ph, Su}, where Su denotes O-sulphonation, which can only modify the amino acid residue Y, and Ph denotes O-phosphorylation, which can only modify the amino acid residues S, T, and Y. Further suppose a practitioner wanted to verify whether this PTM set {Ph, Su} is compatible with the amino-acid residue sequence YIPGTK. If Ph is considered first and matched with Y, then no match is available for Su. Alternatively, if Su is considered first, it would match with Y, and Ph can then be matched with T, leading to the correct conclusion that the PTM set {Ph,Su} and the amino acid sequence YIPGTK are in fact compatible.

Because simply enumerating all possible matchings is likely to be unacceptably slow in many cases, the above example illustrates the need for a systematic analysis of possible matches. Accordingly, various embodiments herein provide a constructive, time-efficient solution based on graph theory.

Reference will now be made to various embodiments of the present teachings. While the present teachings will be described in conjunction with various embodiments, it will be understood that they are not intended to be limiting. On the contrary, the present teachings are intended to cover alternatives, modifications, and equivalents, which may be included within the present teachings.

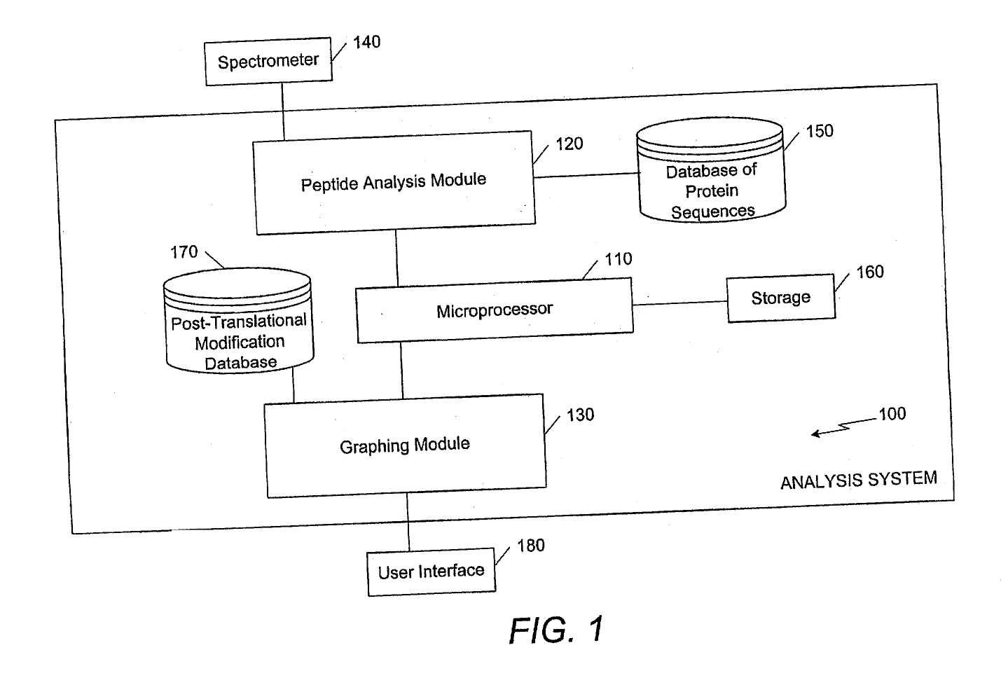

FIG. 1 illustrates an overview of an analysis system 100, in accordance with various embodiments, used to compare the molecular weight of an actual digested peptide fragment with a corresponding theoretical peptide fragment, select a potential PTM set to account for any weight difference, and verify the selected PTM set's compatibility with the theoretical peptide fragment.

The analysis system 100 can be a typical computer apparatus and can include, for example, a motherboard, computer hardware, and software. The motherboard can include a central processing unit (CPU), a basic input/output system (BIOS), one or more RAM memory devices, one or more ROM memory devices, mass storage interfaces which connect to magnetic or optical storage devices such as hard disk storage, and 1 or more floppy drives or removable drives such as CD or DVD. The system 100 can also include, for example, serial ports, parallel ports, USB ports, IEEE 1394 ports and expansion slots. The modules and databases of the analysis system 100 operate in conjunction with a microprocessor 110 which manages data flow and analysis. Any available microprocessor can be used herein, including an Intel Pentium®, Intel Celeron® or AMD® microprocessor, for example.

The analysis system 100 can be an IBM-compatible personal computer, running any of a variety of operating systems including MS-DOS®, Microsoft® Windows®, Linux® or Lindows™. Alternatively, the modules may run on other computer environments, including mainframe systems such as UNIX® and VMS®, or the Macintosh® personal computer environment.

One skilled in the art will recognize that these elements need not be connected in a single unit such as personal computer or mainframe, but may be connected over a network or via telecommunications links. The computer hardware described above may operate as a stand-alone system, or may be part of a local area network, or may include a series of terminals connected to a central system.

The analysis system 100 can include on or more modules and databases that interact with a user interface 180. In various embodiments, a user interface 180 can include, for example, a display monitor, a printer, a keyboard, and/or a mouse or trackball (not shown). The user interface 180 allows the user to control and or modify modules and databases within the analysis system 100. Furthermore, the user interface 180 receives data output from the analysis system 100, allowing the user to receive the analysis.

A mass spectrometer 140 is connected to and sends mass spectrum data to the analysis system 100 after analyzing digested peptide fragments from a protein. In general, the spectrometer 140 is an instrument which separates molecular fragments according to mass by passing them in ionic form through electric and magnetic fields. The spectrometer 140 detects these fields and converts the data into a mass spectrum, which can be used to find a specific peptide's chemical formula, chemical structure, and molecular mass. Any type of mass spectrometer can be used with the methods and systems described herein, including, but not limited to, spectrometers capable of liquid chromatography-mass spectrometry (LC/MS), liquid chromatography-tandem mass spectrometry (LC/MS/MS), gas chromatography-mass spectrometry (GC/MS), and gas chromatography-tandem mass spectrometry (GC/MS/MS). Exemplary spectrometers useful in connection with the teachings herein include, among others, the API 150, API 2000, API 3000, API 4000, API QSTAR, Q TRAP, Voyager, and Applied Biosystems 4700, available from Applied Biosystems (Foster City, Calif.).

The peptide analysis module 120, within the analysis system 100, includes software capable of spectral analysis. More specifically, the software is capable of performing sequencing, peptide mapping and peptide mass fingerprinting, and making other biologically relevant calculations. The peptide analysis module can be configured to form an integrated set of data processing tools for the identification and characterization of peptides.

In some embodiments the peptide analysis module can further integrate utilities that calculate the molecular weight of a peptide fragment. In other embodiments, the peptide analysis module can access a data dictionary. Such dictionaries, contain chemical information such as elements, amino acids, modifications, digest agents and nucleic acids and allow users to easily define modifications, adducts, and cleavage agents. One skilled in the art will note that data dictionaries are often stored in databases. Still other embodiments completely integrate utilities and data dictionaries and automate the data analysis by first determining peptide molecular weights, and then calling upon integrated mapping, sequencing and fingerprinting tools to identify proteins, sequence proteins and identify peptides and partial sequence tags. The results of this analysis can be summarized in results tables and associated reconstructed spectra, which can then be used for higher-order analyses such as, more sophisticated forms of peptide mapping and sequencing which provide additional evidence for protein identification

In various embodiments, the peptide analysis module 120 can be incorporated with a plurality of the aforementioned features. Exemplary software that includes one or more os such features, among others, includes but is not limited to PepMAPPER (available from UMIST, UK), BioAnalyst™ software (available from Applied Biosystems, Foster City, Calif.), Mascot™ (available from Matrix Science, London), PepSea™ (available from Protana, Denmark) or PeptideSearch (available from EMBL, Heidelberg). The above listed software and other relevant software useful in characterizing proteins and peptide fragments can be used according to the methods and systems provided herein. In various embodiments, one or more of the present teachings are embodied in software programs such as those just listed above.

After receiving the mass spectrum data for the peptide fragments from the spectrometer 140, the peptide analysis module 120 calculates the weight of the peptide fragments. After this analysis, the peptide analysis module 120 looks for correspondence between the masses of the peptide fragments and the masses of reference peptides associated. The term “correspondence” when used in the context of two peptide fragments signifies that a reference peptide fragment has the same amino acid sequence as a peptide fragment of interest. The masses of the theoretical peptides and if available, the sequence of the corresponding reference protein from which they originated are stored in the database of protein sequences 150. This database contains many such reference proteins and their corresponding theoretical peptides. The database of protein sequences 150 is a storage site containing a library of reference protein and peptide sequences that can be used by the peptide analysis module 120 for comparison to analyte peptide fragments. In various embodiments, the database of protein sequences 150 also includes a data dictionary which, as mentioned earlier, contains chemical information useful for the determination of biologically relevant calculations.

After receiving data on the corresponding reference peptide fragments from the database of protein sequences 150, the peptide analysis module 120 calculates the molecular weight difference between the analyte and theoretical peptide fragments. After the molecular weight difference has been calculated, the peptide analysis module 120 sends this data to the storage site 160.

The storage site 160 receives the molecular weight difference data from the peptide analysis module 120. The storage site 160 can be, for example, any site capable of holding electronic memory, such as RAM.

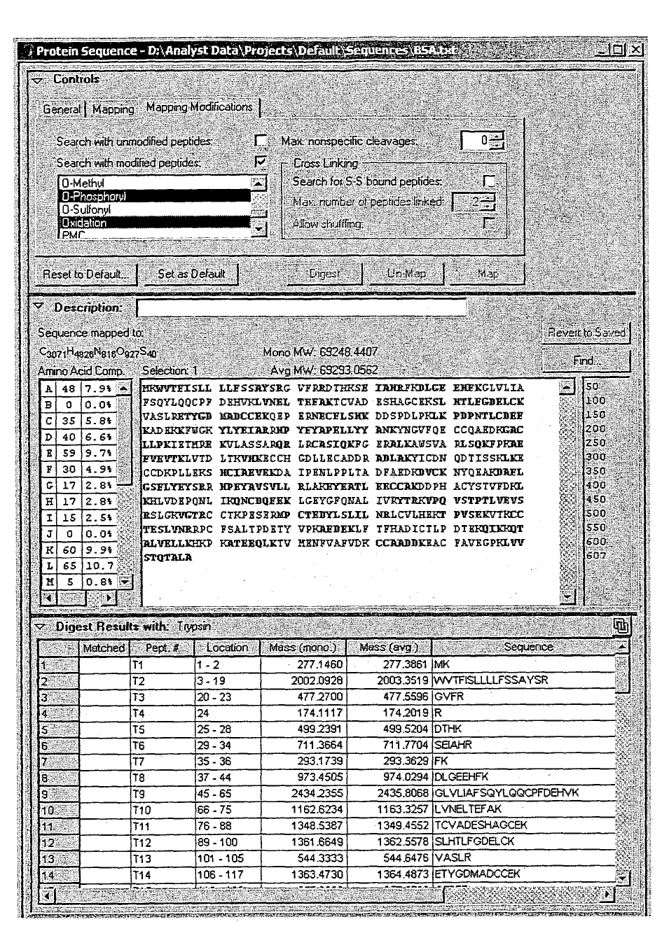

A graphing module 130 can include software capable of selecting and receiving data on the weight difference between the analyte and theoretical peptide fragment from the storage site 160. In various embodiments, in addition, the graphing module can receive information denoting the sequence of the theoretical peptide fragments. Also, the software in the graphing module 130 can select and receive a potential PTM set from the post-translational modification database 170 based on the weight difference data received from the storage site 160. As there can be more than one PTM set that can account for the difference in mass between the analyte peptide and the theoretical peptide, a list of PTM sets can be formed by first allowing the user to specify which PTMs should be considered. In various embodiments this can be achieved by a user interface as shown in FIG. 8. In some embodiments, the members of the list (shown in the upper left hand corner) could comprise a general list that have not been prescreened for chemical compatibility with the amino acids of an amino acid chain of interest (eg. a peptide). In other various embodiments, the list can be prescreened so that the members are known to be chemically compatible with the amino acids of an amino acid chain of interest (eg. a peptide). The graphing module can then form one or more PTM sets that could account for the difference in the mass.

In various embodiments, the graphing module 130 includes software capable of constructing graphs and determining maximum cardinality matching. The graphing module 130 can use graph theory to determine whether the selected post-translational modification set is compatible with the amino acid sequence of the theoretical peptide fragment. One skilled in the art will appreciate that there are several methods of performing maximum cardinality matching one of which uses the augmenting path algorithm. If the PTM set is compatible with the amino acid sequence of the theoretical peptide, the data can be sent to a storage site 160, which can be accessed by the user interface 180. If the PTM set is not compatible with the amino acid sequence of the theoretical peptide, the graphing module 130 can select and receive another potential PTM set from the post-translational modification database 170.

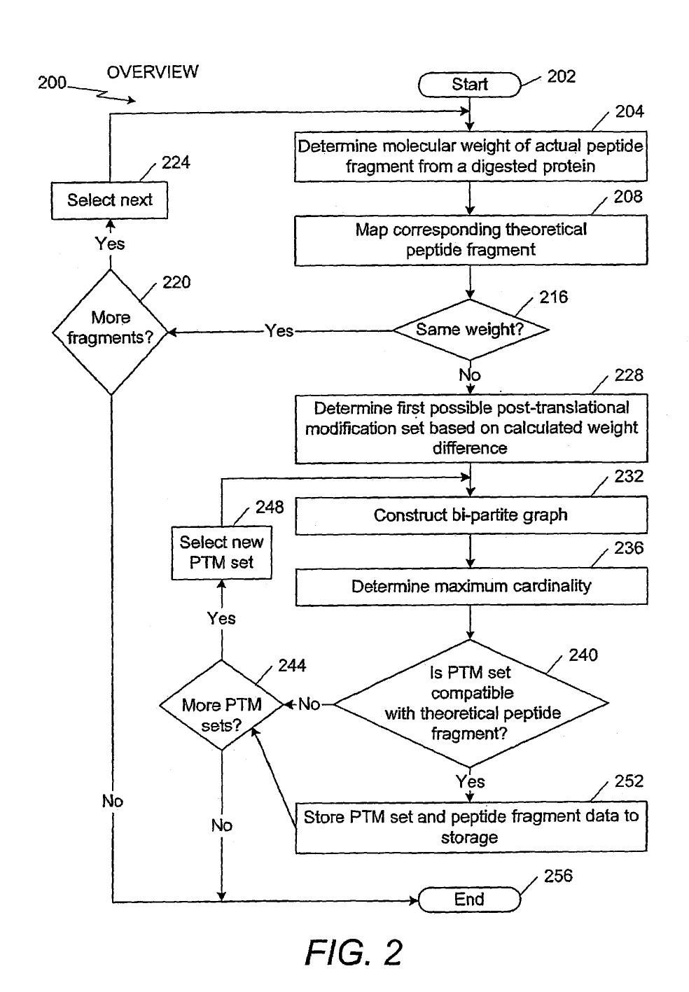

FIG. 2 is a flowchart illustrating an overview of a method, according to various embodiments, for comparing the molecular weight of an experimental peptide fragment with a corresponding reference peptide fragment, selecting a potential PTM set to account for any weight difference, and verifying the PTM set's compatibility with the reference peptide fragment. The process 200 begins at a start state 202 and then proceeds to state 204 where the molecular weight of a peptide fragment from a digested protein is determined. The digested protein can either be known prior to digestion or its identity can be ascertained via peptide mass fingerprinting.

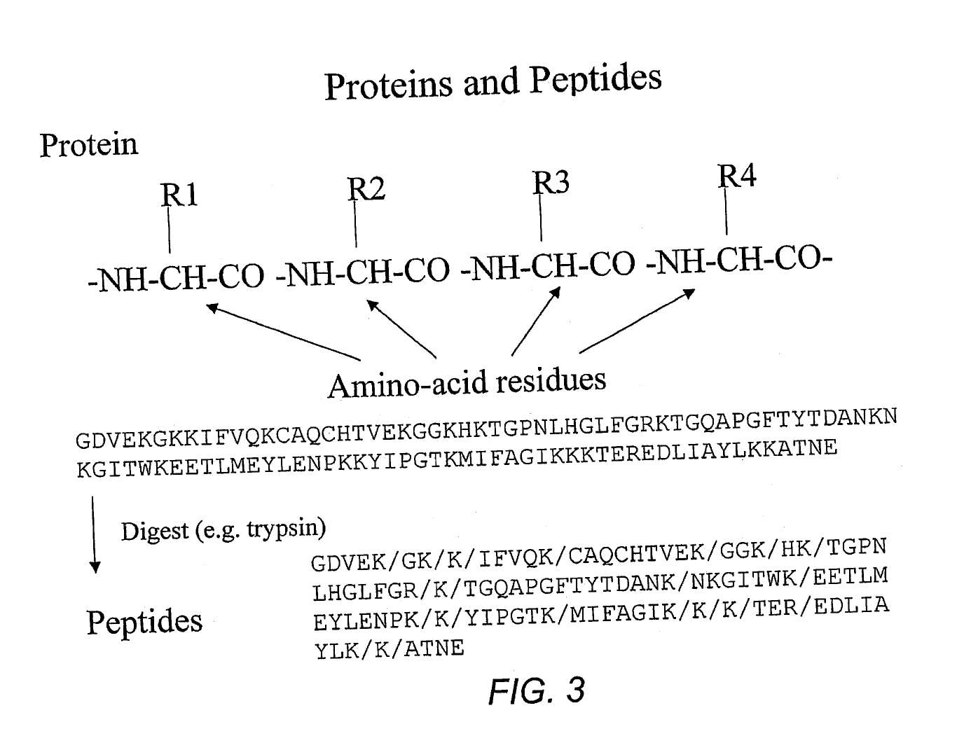

State 204 involves digesting a protein by a suitable means, such as by a protease, e.g., trypsin or pepsin or other protease. FIG. 3 illustrates an example of a protein digest using the protease trypsin. The digested peptide fragments then undergo mass spectrometry in a spectrometer 140. According to various embodiments, the general process of mass spectrometry can include one or more of the following. Peptide fragments are first vaporized and ionized; the ions are accelerated by an electric field and then deflected by a magnetic field into a curved trajectory, which depends on their mass and charge. The ions are then detected photographically or electrically as a mass spectrum. A mass spectrum includes a series of peaks, each corresponding to a different ion. Accordingly, the mass spectrum of a peptide fragment can then be used to find its formula, chemical structure, and molecular mass. Any type of mass spectrometry can be used with the methods and systems described herein, including, but not limited to, liquid chromatography-mass spectrometry (LC/MS), liquid chromatography-tandem mass spectrometry (LC/MS/MS), gas chromatography-mass spectrometry (GC/MS), and gas chromatography-tandem mass spectrometry (GC/MS/MS).

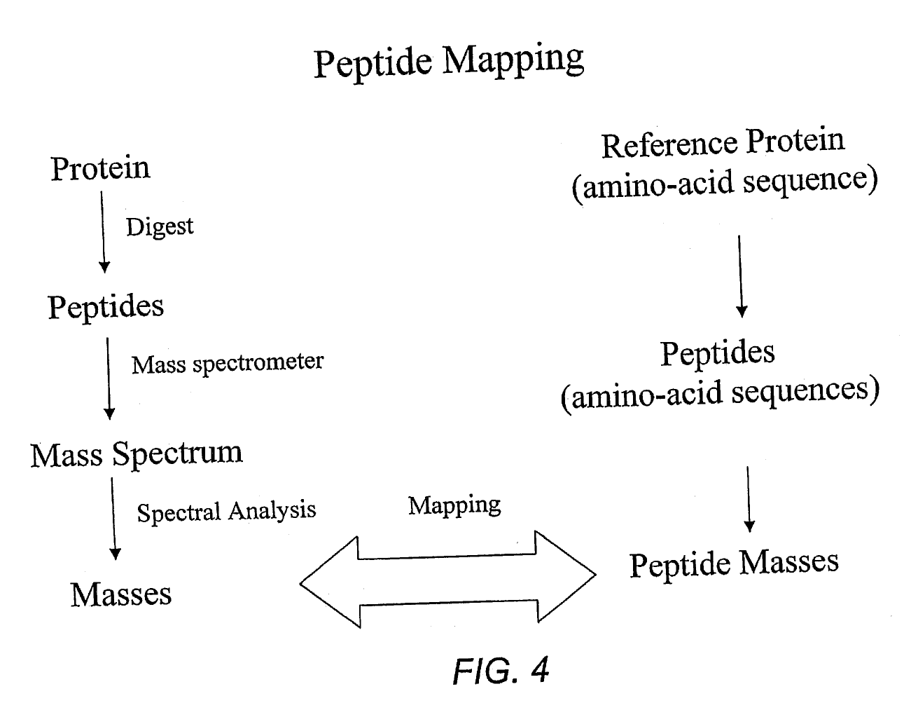

Still in state 204, the peptide analysis module 120 receives the resulting mass spectrum data and undergoes a spectral analysis, utilizing software to determine the analyte peptide fragment's molecular weight. Exemplary commercially available programs capable of such are, Analyst® QS (available from Applied Biosystems, Foster City, Calif.), and Millenium®32 (available from Waters, Milford, Mass.). In various embodiments, utilities can convert an elemental and amino acid composition to mass and vice-versa. This function can be useful, for example, for computing amino acid substitutions to account for an observed mass difference, and calculating masses from a multiple charged ion series or isotope distribution. Notably, such a utility can calculate the molecular weights of post-translational modifications. After the molecular weight of the analyte peptide fragment has been calculated, the process 200 continues to a state 208, where corresponding reference peptide fragments are mapped to the experimental peptide fragments in the peptide analysis module 120. In general, simple peptide mapping involves comparing molecular masses determined by mass spectrometry on a digest of an analyte protein with possible peptide masses from a theoretical reference protein. FIG. 4 provides a broad overview of one method of peptide mapping.

To undergo peptide mapping the peptide analysis module 120 selects a theoretical peptide fragment with the same amino acid sequence as the analyte peptide fragment from the database of protein sequences 150. This determination can be made, for example, based on a comparison of molecular weight. In one embodiment, a theoretical protein that corresponds to the structure of the known analyte protein that has been digested is selected, and undergoes a virtual digest based upon the digestion pattern of the protease that was used in the actual digest. In another embodiment, the protein may not be known and is to be identified via peptide mass fingerprinting. The theoretical protein may be specified as a sequence of standard amino-acid residues, with respect to which the protein actually studied may be chemically modified. These modifications usually take place either during or after translation. In some embodiments, a sequence mutation could also be modeled as a modification, bearing in mind that a mutation may also change the digestion pattern in the sequence.

In various embodiments, the peptide mapping functions embodied in software correlates an analyte peptide's molecular mass, derived from the mass spectrum data, to a corresponding theoretical peptide mass derived from a virtual protein digest. In other embodiments, the mapping software automatically determines peptide molecular weights and then utilizes integrated mapping and sequencing tools to find modifications, sequences or partial sequence tags. Still in other embodiments of mass fingerprinting, multiple proteins are simply and quickly mapped to the data set and modifications from a data dictionary can be added or deleted. In some embodiments, the software maps and displays the raw and deconvoluted spectra and summarizes the mapping and/or fingerprinting results in a table. Peptide mass fingerprinting can be accomplished using a variety of available software, including, for example, with PepMAPPER (available from UMIST, UK), Mascot™ (available from Matrix Science Ltd., London), BioAnalyst™ software (available from Applied Biosystems, Foster City, Calif.), PepSea™ (available from Protana, Denmark) or PeptideSearch (available from EMBL, Heidelberg).

After calculating the molecular weight difference between the analyte and the theoretical peptide fragment, the process 200 reaches a decision state 216. In decision state 216, the peptide analysis module 120 determines whether the actual and theoretical peptides have the same molecular weight. If the peptides have the same molecular weight, the process 200 continue from decision state 216 to another decision state 220 to determine if there are more analyte peptide fragments to analyze from the protein digest. Alternatively, if the peptide analysis module 120 determines in decision state 216 that the actual and theoretical peptide fragments have different molecular masses, the process 200 continues to state 228.

Describing first the situation where both the theoretical and analyte peptide fragments have the same molecular weight, the peptide analysis module 120, in decision state 220, determines whether there is more mass spectrum data from analyte peptide fragments. If there is no more mass spectrum data available, the process 200 proceeds to the end state 256. Alternatively, if the peptide analysis module 120 determines there is more mass spectrum data for additional analyte peptide fragments, the process 200 proceeds to state 224 where the mass spectrum data for the next analyte peptide is selected by the peptide analysis module 120. Once selected, the process 200 returns to state 204 where the peptide analysis module 120 sequences and determines the molecular weight of the analyte peptide fragment based on the mass spectrum data.

Referring back to decision state 216, if the peptide analysis module 120 determines that the theoretical and the analyte peptide fragments have different molecular masses, the process 200 continues to state 228, where the molecular mass difference and amino acid sequence of the theoretical peptide fragment is forwarded to the storage site 160. A graphing module 130 selects and receives data on the amino acid sequence of the theoretical peptide fragment and the molecular mass difference calculation from the storage site 160.

After receiving this data, the graphing module 130 selects and receives a first post-translational modification (PTM) set from the PTM database 170. The PTM database 170 is a storage site containing data on numerous potential peptide post-translational modifications and their corresponding molecular weight. Based on the molecular weight difference data received from the storage site 160, the graphing module 130 selects a potential PTM set from the PTM database 170 that can account for the weight difference between the theoretical and analyte peptide fragment.

Any particular PTM to the peptide fragment causes a predictable shift in the mass distribution of the peptide. Accordingly, an observed shift can be used to infer the possible existence of a set of PTMs. Typically, modifications occur only on amino acids that meet specific requirements, such as having a particular side-chain chemistry or a particular sequence location, for example. Thus it can be desirable to check the compatibility of the selected PTM set with the amino-acid sequence. According to the embodiments described herein, graph theory can be used to verify compatibility.

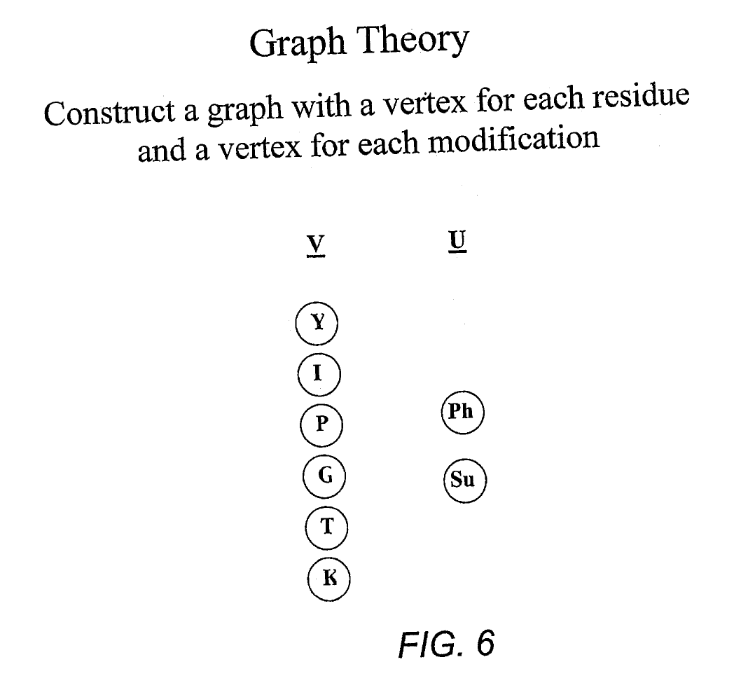

Accordingly, after an appropriate PTM set is selected, the process 200 continues to state 232. In state 232 the graphing module 130 uses software to construct a bipartite graph with two groups of vertices. One group of vertices (U) contains each modification of the selected PTM set and the other group of vertices (V) contains each amino acid of the theoretical peptide fragment. Alternately, the V vertices may be configured to contain only amino acids from the theoretical peptide fragment that can accept at least one modification from the selected PTM set. In a non-limiting example, FIG. 6 illustrates a bipartite graph that can be used to verify whether the PTM set {Ph,Su} is compatible with the amino acid sequence YIPGTK (Tyrosine, Isoleucine, Proline, Glycine, Threonine, Lysine). The lines connecting the modifications represent edges. For purposes herein, edges are only allowed to connect a vertex from group V to a vertex in group U.

From state 232 the process 200 proceeds to state 236 where the graphing module 130 finds a maximum cardinality matching in the constructed graph. Essentially this signifies that the graphing module 130 attempts to match each modification from the U group of vertices with a compatible and unshared amino acid from the V group of vertices. This is accomplished by constructing an edge for every acceptable residue-modification pairing. In constructing the edges, the graphing module 130 adheres to pairing rules. Such rules include, for example, that no amino acid residue can accept more than one modification, and each modification may only be applied to a specific set of amino acid residues.

A matching in the graph module 130 signifies that each constructed edge is connected to only one modification and only one amino acid residue. In other words, each amino acid residue is not paired with more than one modification, and each modification is not paired with more than one amino acid residue. Maximum cardinality matching is achieved when no more edges can be added to the matching. In other words, there are no more unpaired and compatible amino acid residues available to be matched with a modification.

Those skilled in the art will appreciate that there are numerous algorithms available to find a maximum cardinality matching. For example, matching algorithms using an augmenting path search method can be found in Papadimiturou & Steiglitz Combinatorial Optimization: Algorithms and Complexity (1984). A “path” is a sequence of contiguous edges (v₁, v₂), (v₂, v₃), . . . , (vₖ, vₖ₊₁), that is, a sequence of edges 1 . . . k such that every adjacent pair i, i+1 of edges shares a vertex. An “augmenting path” is defined with respect to a matching M, and is a sequence of contiguous edges 1 . . . 2n+1 such that the (n+1) odd edges 1,3, . . . ,2n+1 are not in M, while the n even edges 2,4, . . . ,2n are in M, and the first and last vertices are not incident upon any edge in M. Note that the path contains n edges in M and n+1 not in M.

Given an augmenting path, a new edge set, consisting of all the odd edges of M, can be constructed. The new set is also a valid matching, because by construction no vertex is shared; furthermore it contains one extra edge. Thus, an augmenting path allows a new matching with cardinality one greater to be constructed. After a new matching is constructed, the graphing module searches the graph for another augmenting path to allow for a new match. Generally the augmenting process can be described as follows. The graph begins with any match M (usually a graph without any edges). Next the graph is searched for an augmenting path with respect to M. If found, M is augmented (another edge is drawn) and the graph is searched again for another augmenting path. This process continues until no more augmenting paths can be found.

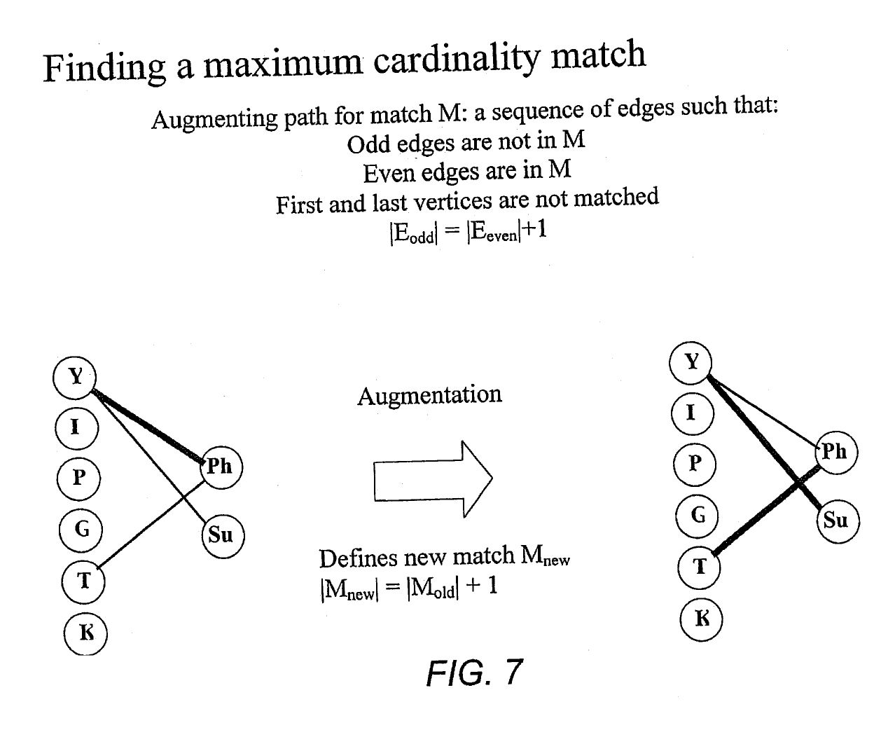

FIG. 7 illustrates the above-described method of finding a maximum cardinality match using an augmenting path. Referring to the bipartite graph on the left, the algorithm will connect modifications {Ph,Su} to their acceptable amino acids from the peptide YIPGTK. This connection will form a continuous path of edges. The darkened edge connecting the modification Ph to the amino acid residue Y is an even edge (the 2^{nd} edge) and is therefore included in the first match (M_{old}). In contrast, the lighter edges connecting Su to Y and Ph to T are odd edges (the 1^{st} and 3^{rd} edge) and are therefore excluded from the first match (M_{old}).

Now referring to the graph on the right of FIG. 7 (M_{new}). After an augmenting path is found, the graph now contains two edges which are indicated by the darker edges connecting Su to Y and Ph to T. It will be appreciated that this graph still represents a match because each constructed edge is connected to only one modification and only one amino acid residue. It will further be appreciated that this is a maximum cardinality match because no more edges can be added to the matching. Further, because there are two edges in the maximum cardinality match and there are two modifications in the modification set {Ph,Su,}, the set is compatible to the peptide.

In various embodiments, an algorithm based on an augmenting path and implemented into a computer program can be used to find a maximum cardinality matching. Those skilled in the art will appreciate that other algorithms, which can be faster than augmentation can be used to find a maximum cardinality matching. In various embodiments, the asymptotically faster algorithm described in Papadimiturou & Steiglitz (1984) can be used to find maximum cardinality matching in state 236. After a maximum cardinality matching has been achieved, the process 200 proceeds to the decision state 240. In decision state 240, the graphing module 130 checks to verify that the number of edges in the graph is equal to the number of modifications in the selected PTM set. If in decision state 240, the graphing module's 130 calculation signifies that there are fewer edges than modifications, the PTM set is not compatible with the theoretical peptide fragment. Accordingly, the process 200 continues to decision state 244 where the graphing module 130 assesses whether there are more potential PTM sets from the PTM database 170 that could account for the molecular weight difference between the theoretical and analyte peptide fragments. If no more PTM sets can account for the molecular difference between the theoretical and analyte peptide fragments, the process 200 continues to an end state 256. If however there are more PTM sets available to account for the molecular difference, the graphing module 130 will select a new PTM set from the PTM database 170 in state 248. After selecting a new PTM set, the process 200 will return to state 232, where the graphing module 130 will construct a new graph.

Alternatively, if in decision state 240, the graphing module 130 calculates that the number of edges is equivalent to the number of modifications, the selected PTM set is compatible with the particular theoretical peptide fragment. Once compatibility is confirmed, the process 200 continues to state 252 where the PTM set along with the data on the compatible theoretical peptide fragment are sent to a storage site 160. From state 160 the process continues to decision state 244 where the graphing module 130 assesses whether there are more potential PTM sets from the PTM database 170 that could account for the molecular weight difference between the theoretical and analyte peptide fragments. This function is particularly useful when there are multiple potential PTM sets that can account for the weight difference between the actual and theoretical peptide fragments. A user can view any type of mass fingerprinting or peptide mapping result from the user interface 180. Fingerprinting and mapping results can include, for example, the name of the protein sequence file, peptides which match the N- and C-terminal rules for the digest agent, the peptide number from the digest results for the linked sequence, the location of the mapped peptide in the sequence, the calculated molecular weight of the mapped peptide, the difference between the calculated molecular weight and the mass in the analysis table, the sequence of the mapped protein, the sequence of the analyte peptide fragments, post-translational modifications and the location of the PTMs.

One skilled in the art will appreciate that the graphing module and its functionality 130 can be incorporated into the peptide analysis module 120 thus forming a highly integrated system for peptide mass fingerprinting and peptide mass mapping.

It will be appreciated that, among various advantages, the teachings herein can provide a systematic, flexible, and computationally efficient way of checking compatibility of possible chemical modifications inferred from mass spectrometric data on proteins and peptides.

All publications and patent applications referred to herein are hereby incorporated by reference to the same extent as if each individual publication or patent application was specifically and individually indicated to be incorporated by reference.

Those of ordinary skill in the art will clearly understand that many modifications are possible in the above embodiments without departing from the teachings thereof. All such modifications are intended to be encompassed herein.

What is claimed is:

1. A method for use in peptide mass mapping to identify post-translational modifications, comprising:
measuring the molecular weight of a peptide fragment;
comparing that measured molecular weight to a molecular weight expected for an unmodified fragment having the same sequence, thereby ascertaining a difference from an unmodified fragment;
determining one or more sets of post-translational modifications that could account for said difference in the measured molecular weight of said peptide fragment and said unmodified fragment; and
applying a graph theory formulation to determine chemical compatibility between the measured molecular weight and a set of possible post-translational modifications.

2. The method of claim 1, wherein said graph theory formulation includes maximum cardinality matching in a bipartite graph.

3. A method to determine compatibility between an amino-acid residue chain having an experimentally-ascertained molecular weight and a known amino acid sequence and a set of post-translational chemical modifications, comprising:
constructing a bipartite graph comprising a vertex for each residue, a vertex for each modification, and an edge for each compatible pair; and
seeking a maximum cardinality matching comprising a set of edges (i) wherein no two edges share a vertex, and (ii) wherein every modification is paired with a residue.

4. A method to determine chemical compatibility of an amino-acid residue chain with a set of chemical modifications, comprising: constructing a graph, finding a maximum cardinality matching, and determining whether the cardinality is equal to the number of modifications.

5. The method of claim 4, wherein said maximum cardinality matching is found by selecting any matching, finding an augmenting path, using this to define a new matching, and repeating this process until no additional path can be found.

6. A method for peptide analysis, comprising:
comparing a measured mass of an analyte peptide against the masses of theoretical peptides derived from a reference protein; and
applying a graph theory formulation to determine the chemical compatibility between a selected set of post-translational modifications (PTMs) with the theoretical peptides;
whereby a set of candidate peptides is developed, comprising one or more peptides, including one or more peptides bearing one or more PTMs, having a mass consistent with that of said analyte peptide.

7. The method of claim 6, wherein said measured mass of said analyte peptide is determined by mass spectrometry of a protein digest.

8. A program storage device readable by a machine, embodying a program of instructions executable by the machine to perform method steps for peptide analysis, said method steps comprising:
(i) comparing a measured mass of an analyte peptide against the masses of theoretical peptides derived from a reference protein; and
(ii) applying a graph theory formulation to determine the chemical compatibility of a selected set of post-translational modifications (PTMs) with the theoretical peptides;
whereby a set of candidate peptides is developed, comprising one or more peptides, including one or more peptides bearing one or more PTMs, having a mass consistent with that of said analyte peptide.

9. The device of claim 8, wherein said graph theory formulation includes maximum cardinality matching in a bipartite graph.

10. A program storage device readable by a machine, embodying a program of instructions executable by the machine to perform method steps for use in peptide analysis, said method steps comprising:
applying a graph theory formulation to determine chemical compatibility of an amino-acid residue chain with a set of chemical modifications.

11. The device of claim 10, wherein said graph theory formulation includes maximum cardinality matching in a bipartite graph.

12. The device of claim 10, wherein said chemical modifications comprise post-translational modifications.

13. The device of claim 10, wherein said method steps further comprise:
providing output relating a measured peptide mass with a theoretic peptide having some chemical modification or set of chemical modifications.

14. A method in a computer system for analysis of an analyte peptide, comprising:
receiving an input comprising a mass of an analyte peptide;
presenting to a user a listing comprising a plurality of post-translational modifications (PTMs); and
receiving from said user a user-selected set derived from said plurality of PTMs;

15. The method of claim 14 further comprising determining one or more sets of post-translational modifications wherein each set comprises one or more post-translational modifications and each set can account for said mass difference within a defined mass tolerance.

16. The method of claim 15 further comprising presenting to said user one or more theoretical peptides, bearing one or more PTMs from said user-selected set that have been checked for chemical compatibility with said theoretical peptides, having a mass matching that of said analyte peptide within a defined mass tolerance.

17. The method of claim 14, wherein said chemical compatibility check is by way of a graph theory formulation.

18. In a graphical user interface, a method for permitting a user to select set of candidate post-translational modifications comprising:
presenting to a user a listing comprising a plurality of post-translational modifications (PTMs); and
receiving from said user a user-selected set derived from said plurality of PTMs.

19. A method to select a set of candidate post-translational modifications based on the difference of a measured parameter between an analyte peptide and a theoretical peptide comprising:
measuring a parameter of an analyte peptide;
computing the same parameter as in the previous step for a corresponding theoretical peptide;
computing a difference between the measured parameter of the analyte peptide and the computed parameter of the theoretical peptide;
selecting from a database of post-translational modifications one or more post-translational modifications that could account for said difference; and
reporting the set.

20. The method of claim 16 where the measured parameter is mass.

21. A program storage device readable by a machine, embodying a program of instructions executable by the machine to perform method steps for use in selecting a set of candidate post-translational modifications comprising:
measuring a parameter of an analyte peptide;
computing the same parameter as in the previous step for a corresponding theoretical peptide;
computing a difference between the measured parameter of the analyte peptide and the computed parameter of the theoretical peptide;
determining one or more sets of post-translational modifications that could account for said difference in the measured molecular weight of said peptide fragment and said unmodified fragment; and
reporting the one or more sets.

22. The device of claim 21 where the measured parameter is mass.

23 A method for use in peptide mass mapping, comprising:
applying a graph theory formulation to determine chemical compatibility of an amino-acid residue chain with a set of chemical modifications.

24. The method of claim 23, wherein said graph theory formulation includes maximum cardinality matching in a bipartite graph.

25. A system for analyzing proteins or peptides, comprising:
an input portion for receiving peptide mass data;
a database of protein sequences;
a peptide analysis module adapted for communication with said input portion and with said database of protein sequences;
a microprocessor adapted for communication with said peptide analysis module;
a database of post-translational modifications;
a graphing module adapted for communication with said microprocessor and with said database of post-translational modifications; and
an output portion, adapted for communication with said graphing module.

26. The system of claim 25, further comprising a user interface adapted for communication with said output portion.

27. The system of claim 25, further comprising a mass spectrometer adapted for communication with said input portion.

28. The system of claim 25, further comprising a storage component, adapted for communication with said microprocessor.

29. The system of claim 25, wherein said graphing module is configured to apply a graph theory formulation to determine chemical compatibility of an amino-acid residue chain with a set of chemical modifications.
